(19) United States
(12) Patent Application Publication
(10) Pub. No.: US 2006/0171890 A1
(43) Pub. Date: Aug. 3, 2006

(54) Methods for evaluating the activity of candidate agents

(76) Inventors: David C. Yeomans, Stanford, CA (US); Martin Angst, Stanford, CA (US); Michael C. Peters, Stanford, CA (US); Mary Peters, Hobart, IN (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/342,246

(22) Filed: Jan. 27, 2006

Related U.S. Application Data
(60) Provisional application No. 60/647,949, filed on Jan. 28, 2005.

(51) Int. Cl. A61K 49/00

(52) U.S. Cl. 424/9.2

(57) ABSTRACT

Methods for evaluating the activity of candidate agents are provided. In practicing the subject methods, a candidate agent is administered to an injured tissue site of an animal and the activity of the candidate agent on the injured tissue site is evaluated. Also provided are kits and systems for use in practicing the subject methods.

### CROSS-REFERENCE

This application claims the benefit of U.S. Provisional Application No. 60/647,949, filed Jan. 28, 2005, which application is incorporated herein by reference.

### BACKGROUND OF THE INVENTION

### Background

Despite years of therapeutic advances, acute and chronic pain remain major medical problems. Acute pain following surgical procedures has been associated with adverse physiological alterations in the pulmonary (Ford G. T., et al: Am Rev Resp Dis 127:431, 1983), cardiovascular (Ready B: In Anesthesia, 3rd edition, Miller R D (ed), Churchill Livingstone, New York, 1990 pp. 2135-2146), gastrointestinal, urinary (Cousins M: In Textbook of Pain, 2nd Edition: Wall, Meizack (eds): Churchill Livingstone, New York, 1989, pp 284-305) and neuroendocrine systems (Kehlet H: In Acute Pain Management. Cousin and Phillips (eds): Churchill Livingstone, New York, 1986, pp 49). Many of these undesirable physiological changes can be minimized with effective analgesia (Cousins M: In Textbook of Pain, 2nd Edition. Wall, Melzack (eds): Churchill Livingstone, New York, 1989, pp 284-305).

Currently, most pharmaceutical companies rely on animal models to examine the importance of particular target molecules in pain and inflammation and the effectiveness of a candidate analgesic, local anesthetic, or anti-inflammatory agent in alleviating or preventing pain or inflammation. However, results obtained in animal models may not be as useful as believed since the activity witnessed in these models may not correlate to activity in humans. For example, it is not definite whether or not the candidate agent will have a toxic effect on the human subject.

The problems posed in development of novel analgesic drugs are further exacerbated by the fact that it typically takes many years of animal research before such novel molecules can even be administered to human subjects. Even then, problems are posed in the method of delivering the candidate analgesic to the human. Currently, the candidate is delivered in a systemic fashion, even though the injury may be localized to one specific location. As a result, introduction of technologies that significantly improve the efficiency and accuracy of the research process, while also reducing the development cost, can be helpful in the development of new therapeutics to treat inflammation and pain.

There is a continued need in the field for new methods for evaluating the effectiveness of candidate pharmaceutical agents, e.g., analgesic, anesthetic, or anti-inflammatory agents, in humans. The present invention addresses this need.

Relevant Literature

U.S. Patent of interest include: U.S. Pat. Nos. 6,030,358; 5,925,018. Published U.S. Applications of interest include: 20030167031. Additional references of interest include: Ungerstedt & Hallstrom, Life Sciences (1987) 41:861-864; Anderson et al., Acta Dermato-Venerologica (1991) 71:389-393; Sauerstein et al., Journal of Physiology (2000) 529.3:803-810

### SUMMARY OF THE INVENTION

Methods for evaluating the activity of candidate pain/inflammation pharmaceutical agents, e.g., anti-inflammatory, local anesthetic, or analgesic agents, are provided. In practicing the subject methods, a candidate agent is administered systemically or locally to a normal or injured tissue site of an animal and the local activity of the candidate agent on the tissue site is evaluated. Also provided are kits and systems for use in practicing the subject methods.

### FEATURES OF THE INVENTION

One feature of the invention provides methods for evaluating the activity of a candidate pain/inflammator pharmaceutical agent, e.g., an anti-inflammatory, local anesthetic, or analgesic agent. In practicing the subject methods, a candidate agent is administered systemically or locally to a normal or injured tissue site of an animal and the activity of the candidate agent on the tissue site is evaluated. In such methods the candidate agent may be locally administered, e.g., by microdialysis or plasma phoresis. The effects of candidate agent administration are then assessed on the subject tissue. The animal of such methods may be a mammal, and in many embodiments it is a human. In such methods the candidate agent, e.g., analgesic, local anesthetic, or antiinflammatory agent, may be a small molecule. Such methods may further include injuring the tissue site prior to locally administering the candidate agent. Such injury may include physically or chemically injuring the tissue site. Evaluation of the candidate analgesic agent's activity in such methods may include assaying for the release of at least one pain, inflammation, or injury mediating chemical from the site, such as prostaglandins, cytokines, or neuropeptides; measuring vascular perfusion at the site, such as by Laser Doppler; physical inspection of the injured tissue site; and subjective interrogation of the animal, for example, by quantitative sensory testing (QST).

Another feature of the invention provides kits and systems which include a local administration element for locally administering a candidate agent to a tissue site and instructions for using the element in a method of evaluating the activity of a candidate agent. The kits and systems may further comprise a candidate agent; a local administration element, such as a microdialysis device; cutaneous tissue site injury element, such as a physical injury element or a chemical injury element; a tissue site injury assessment element; and an animal.

### DETAILED DESCRIPTION OF THE INVENTION

Methods for evaluating the activity of candidate agent, e.g., analgesic, local anesthetic, or antiinflammatory agent, are provided. In practicing the subject methods, a candidate agent is administered to normal or injured cutaneous, joint, muscle, or other tissue site of an animal and the activity of the candidate agent on the tissue site is evaluated. Also provided are kits and systems for use in practicing the subject methods.

Before the present invention is described, it is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a candidate analgesic agent” includes a plurality of such candidate analgesic agents and reference to “the injury” includes reference to one or more injuries and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the subject invention, the methods will be described first, followed by a review of representative applications in which the methods find use, as well as kits and systems thereof that find use in practicing the subject methods.

Methods

As summarized above, the subject invention provides methods for evaluating the activity of a candidate agent, such as a pain/inflammation pharmaceutical agent, e.g., antiinflammatoy, local anesthetic, or analgesic agent, on a normal or injured tissue site. By “antiinflammatory” is meant a medication that reduces or eliminates local inflammation of tissue. Accordingly, by “anti-inflammatory activity” is meant effectiveness of the candidate anti-inflammatory agent in reducing or eliminating inflammation. By reducing inflammation is meant at least decreasing inflammation by at least about 30%, such that inflammation is substantially if not completely eliminated. By “local anesthetic” is meant a medication that prevents, reduces or eliminates pain and other sensations induced by local tissue injury, thereby allowing access to the tissue for surgical or other invasive procedures. Accordingly, by “local anesthetic activity” is meant reducing local sensation by at least 30%, such that sensation is sensation is substantially or completely eliminated. By “analgesic” is meant a medication that reduces or eliminates pain. Accordingly, by “analgesic activity” is meant effectiveness of the candidate analgesic agent in reducing or eliminating pain.

The subject methods may be used to evaluate the activity of a candidate anti-inflammatory, local anesthetic, or analgesic agent on normal or injured tissue site in a variety of different types of subjects. Generally the subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the subjects will be humans.

The activity of a candidate-agent on a tissue site is evaluated according to the subject methods by locally or systemically administering the candidate agent to a tissue site and assessing the site to evaluate the candidate agent's activity in the tissue area being assessed.

In practicing the subject methods, the candidate agent is locally administered to a normal or injured tissue site. By “injured” is meant damaged, distressed, or physically harmed. Accordingly, such injured tissue site may be the result of physical injury or chemical injury to the tissue site. In some embodiments, physical injury will be the result of sunburn. By “sunburn” is meant inflammation or blistering of the skin caused by overexposure to a direct light source, such as sunlight or a heat lamp. In other embodiments, chemical injury will be the result of locally administering to a cutaneous tissue site chemicals, which induce at least one or more of pain and inflammation. Examples of pain-inducing chemical agents include acids, bases, vanilloids (such as capsaicin), mustard oil, adjuvents (such as Freund's complete adjuvant), fixatives (such as formalin).

The candidate agent, e.g., anti-inflammatory, local anesthetic, or analgesic agent, may be a one or a mixture of a variety of different compounds, including: polypeptide, polynucleotide, including antisense oligonucleotides, and naturally occurring or synthetic small molecule compounds.

In certain embodiments, the candidate agent administered to the tissue site is a naturally occurring or synthetic small molecule compound, which includes numerous chemical classes, though typically they are organic molecules, preferably small organic compounds having a molecular weight of more than 50 and less than about 2,500 daltons. Candidate agents include functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs, or combinations thereof.

In other embodiments, the candidate agent administered to the injured cutaneous tissue site is a polypeptide, e.g., proteinaceous, active agents. The proteins may be human proteins or homologs or proteins (or fragments thereof) from other species, i.e., other animal species, where such homologs or proteins may be from a variety of different types of species, usually mammals, e.g., rodents, such as mice, rats; domestic animals, e.g. horse, cow, dog, cat; and primates, e.g., monkeys, baboons, humans etc. By homolog is meant a protein having at least about 35%, usually at least about 40% and more usually at least about 60% amino acid sequence identity to the specific human proteins as identified above, where sequence identity is as measured by the BLAST Compare Two Sequences program available on the NCBI website using default settings.

In certain embodiments, the candidate agent administered to the tissue site is a polynucleotide or nucleic acid composition. The nucleic acid may be coding sequences, e.g., genes, gene fragments etc., which may be present in expression vectors, where such vectors generally have convenient restriction sites located near the promoter sequence to provide for the insertion of nucleic acid sequences. A transcription cassette may be prepared that includes a transcription initiation region, the target gene or fragment thereof, and a transcriptional termination region. The transcription cassette may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

In other embodiments, the candidate agent is an antisense oligonucleotide (ODN), particularly synthetic ODN having chemical modifications from native nucleic acids, or nucleic acid constructs that express such anti-sense molecules as RNA. The antisense sequence is complementary to the mRNA of a targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be used as a candidate analgesic agent, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Alternatively, the candidate agent can also be double-strand RNA molecules (Sharp (1999) Genes and Development 13: 139-141). RNAi, otherwise known as double-stranded RNA interference (dsRNAi) or small interfering RNA (siRNA), has been extensively documented in the nematode C. elegans (Fire, A., et al, Nature, 391, 806-811,1998). In such embodiments, an effective amount of the RNAi agent is administered to the injured cutaneous tissue site. The RNAi molecules are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, 23 bp are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

In practicing the subject methods, the candidate agent is administered systemically or locally to the tissue site. The agent is typically administered to the host in a physiologically acceptable delivery vehicle, e.g., as a pharmaceutical preparation. A variety of representative formulations, dosages, routes of administration for candidate analgesic agents are described below.

The candidate agent of the subject methods is administered to the subject animal to evaluate the agent's activity, e.g., anti-nflammatory, local anesthetic, or analgesic activity. The candidate agent may be administered in convenient formulations, including a pharmaceutical formulation that includes the candidate agent. In general, a formulation comprises an amount of at least one candidate agent as described above.

In the subject methods, the candidate agent may be administered to the host using any convenient means. In practicing certain embodiments of the subject methods, the candidate agent is locally administered to the injured cutaneous tissue site. By “locally administered” is meant administering to a particular location and not systemically. In alternative methods, the candidate agent is administered systemically such that the local effects of systemic administration of the candidate agent on the subject tissue may be assessed.

Thus, the candidate agent can be incorporated into a variety of formulations for local or systemic administration. More particularly, the agent can be formulated into pharmaceutical compositions by combination with appropriate, pharmaceutically acceptable carriers or diluents, and may be formulated into preparations in semi-solid and liquid forms, such as powders, granules, ointments, solutions, and injections.

In pharmaceutical dosage forms, the candidate agents may be administered in the form of their pharmaceutically acceptable salts, or they may also be used alone or in appropriate association, as well as in combination, with other pharmaceutically active compounds. The following methods and excipients are merely exemplary and are in no way limiting.

The candidate agent can be formulated into preparations for local injection or as topical ointment to be administered to the injured cutaneous tissue site by dissolving, suspending, or emulsifying them in an aqueous or nonaqueous solvent, such as vegetable or other similar oils, synthetic aliphatic acid glycerides, esters of higher aliphatic acids, or propylene glycol; and if desired, with conventional additives such as solubilizers, isotonic agents, suspending agents, emulsifying agents, stabilizers, and preservatives.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

An agent of the invention can be administered as injectables or as ointments. Typically, injectable and ointment compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985; Remington: The Science and Practice of Pharmacy, A. R. Gennaro, (2000) Lippincott, Williams & Wilkins. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Conventional and pharmaceutically acceptable routes of administration include intramuscular, subcutaneous, intradermal, topical application, intravenous, and, other parenteral routes of administration. Routes of administration may be combined, if desired, or adjusted depending upon the candidate agent and/or the tissue site. The composition can be administered in a single dose or in multiple doses.

The candidate agent can be administered to a host using any available conventional localized methods and routes suitable for delivery of conventional drugs. In general, routes of administration contemplated by the invention include, but are not necessarily limited to parenteral routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect local delivery of the candidate agent.

Methods of administration of the agent through the skin include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

In some embodiments the candidate agent may be locally administered to the tissue site by topical administration directly to the tissue site as an ointment. In such embodiments the candidate agent will be formulated in a composition with buffers and carriers to allow the agent to be absorbed by the injured cutaneous tissue site.

In other embodiments the candidate agent may by locally administered to the tissue site by injection directly to the tissue site. Injection of the candidate agent may be achieved by intramuscular or intradermal injection. In such embodiments, the candidate agent will be formulated in a composition with acceptable excipients that facilitate stability and absorption of the candidate agent by the surrounding tissue site.

In yet other embodiments local administration of the candidate agent may be achieved by using a microdialysis or plasma phoresis tube inserted at, or near, the tissue site. In such embodiments, the microdialysis or plasma phoresis tube will be inserted within the skin, muscle, joint, or other subject tissue. In such embodiments, the candidate agent will be formulated in a composition with acceptable excipients that facilitate stability and absorption of the candidate analgesic agent by the surrounding tissue site.

In such an embodiment at least one microdialysis or plasma phoresis tube may be used to locally administer the candidate agent by way of diffusion into the tissue site. The number of tubes that may be used will range from 1 to about 10, usually from 1 to about 6, more usually from 1 to about 4. The diameter of the tubes that may be used will range from about 100 micometers to about 1000 micrometers, such as from about 100 micrometers to about 600 micrometers, including from about 200 micrometers to 400 micrometers. The molecular size selectivity of the microdialysis tubes that may be used will range from about 5 kilodaltons to about 20,000 kilodaltons, such as from about 10 kilodaltons to about 10,000 kilodaltons, including from about 20 kilodaltons to about 5000 kilodaltons.

The depth of the insertion and the distance traversed under the skin or in muscle or joint by the microdialysis or plasma phoresis tube will be dictated in large part by the location of the tissue site on the animal, the type of injury sustained, if any, by the tissue site, the size selectivity of the tube, the number of tubes inserted, and the molecular characteristics of the pain and/or inflammatory mediator chemical markers assessed, and the molecular characteristics of the candidate agent. For example, a suitable insertion depth in the skin will range from about 10 micrometers to about 5000 micrometers, such as from about 100 micrometers to about 1000 micrometers, including from about 200 micrometers to about 600 micrometers. A suitable distance traversed in the subject tissue by the tube will range from about 100 micrometers to about 10000 micrometers, such as from about 150 micrometers to about 5000 micrometers, including from about 2000 micrometers to about 4000 micrometers.

In such embodiments, the candidate agent may be delivered to the subject tissue site using the microdialysis or plasma phoresis tubes by manual pressure using a syringe, or alternatively, by using a pump. Any pump available in the art may be used with this embodiment, for example, the Pump 22 Multiple Syringe Pump (Harvard Apparatus, Hollister, Mass.), and the Model KDS 101 Two-Syringe Microdialysis Pump (GeneQ Inc., Montreal, Canada).

In practicing the methods of the subject invention, the location of tissue may be anywhere on the skin, in any muscle, or any joint of the subject animal that facilities the ability to locally sample pain and inflammatory mediating chemicals, and to locally administer the candidate agent and allows for evaluation of the activity of interest, e.g., anti-inflammatory, local anesthetic, or analgesic activity of the candidate agent. Accordingly, the location of tissue may be, for example, but not limited to, the upper arm, lower forearm, shoulder, knee, chest, lower back, and stomach.

Although the amount of candidate agent delivered to the tissue site will vary, a suitable dosage range is one which provides up to about 0.01 μg to about 1,000 μg or about 10,000 μg of the candidate agent. Those of skill will readily appreciate that dose levels can vary as a function of the specific candidate agent, the severity of the injury symptoms and the susceptibility of the subject animal to side effects. Suitable dosages for a given candidate agent are readily determinable by those of skill in the art by a variety of means.

In practicing the subject methods, the activity of the candidate agent, e.g., anti-inflammatory, local anesthetic, or analgesic agent, is evaluated following local administration to subject tissue or by systemic application. As noted above, evaluating the effectiveness of the candidate agent is done by assessing the reduction or elimination of pain or inflammation at the injured cutaneous tissue site. By reducing pain is meant at least decreasing pain or inflammation in the injured tissue site, by at least about 25%, such as at least about 30%, including at least about 40%, or substantially if not completely eliminating pain, such that the pain is not sensed by the animal, chemical markers of pain are eliminated or inflammation or chemical markers of inflammations are eliminated. In evaluating the activity of the candidate agent, the level of pain and inflammation is determined just prior to and following the local or systemic administration of the candidate agent at the injured tissue site.

In some embodiments, the activity of the candidate agent is evaluated by subjectively interrogating the subject animal regarding the injured tissue site just prior to and following the local or systemic administration of the candidate agent at the injured tissue site. Such subjective interrogation may include questions regarding pain sensation and severity felt at the injured tissue site. The interrogation may include, for example, question on the existence of pain, the type of pain, the location of the pain, the severity and intensity of the pain, the localization of the pain, and any other subjective descriptions of the pain the subject animal may convey regarding the injury at the tissue site.

In other embodiments the activity of the candidate agent is evaluated by assaying for the release of pain and inflammatory mediating chemicals prior to and following the local or systemic administration of the candidate agent at the injured tissue site. Pain and inflammatory mediating chemicals may include, for example, polypeptides and fatty acids, such as prostaglandins, cytokines, growth factors, neurotransmitters, or necrosis factors. In such embodiments, pain and inflammatory mediating chemicals may be recovered by, using microdialysis or plasma phoresis tubes. Accordingly, pain and inflammatory mediating chemicals present at the injured tissue site will diffuse into the mixture in the dialysis or phoresis tube and can then be assayed. Once collected, assaying for the pain and inflammation mediating chemicals may be carried out, for example, by any of several immunological based assays known in the art, such as ELISA, immunoprecipitation, BioPlex, western blot, or any biochemical based assays.

In yet other embodiments the activity of the candidate agent is evaluated by measuring vascular perfusion at the injured tissue site prior to and following the local administration of the candidate agent at the injured tissue site. Vascular perfusion at the injured cutaneous tissue site may be measured by using Laser Doppler, for example, by scanning laser Doppler imaging. In such a procedure, Scanning Laser Doppler Imaging can be used to construct a multi-point map of blood flux over a wide area by scanning a low power laser source over the surface of the injured cutaneous tissue site, to build up an image of dermal perfusion. Such measurements may be made prior to and after the local or systemic administration of the candidate analgesic agent to evaluate the activity of the candidate analgesic agent in reducing or eliminating pain or inflammation at the injured tissue site.

Alternatively, one or more of these methods may be combined in order to evaluate the activity of the candidate agent at the injured tissue site.

Utility

The subject methods find use in a variety of different applications in which the evaluation of the activity of a candidate agents, e.g., anti-inflammatory, local anesthetic, or analgesic agents, is required. As such, the subject methods find use in applications of drug development in which evaluation of the activity of a candidate agent is desired. Such applications include determining whether a candidate agent is effective at decreasing or eliminating pain and/or pain mediating chemicals, or inflammation or inflammatory mediators in an injured tissue site of an animal.

Kits

Also provided are kits that find use in practicing the subject methods, as described above. For example, in some embodiments, kits for practicing the subject methods may include a local administration element for locally administering a candidate agent to a cutaneous tissue site. In some embodiments, the local administration element is a microdialysis device. The kits for practicing the subject methods may also include cutaneous tissue site injury element. In some embodiments the cutaneous tissue site injury element is a physical injury element, such an ultraviolet light source. In yet other embodiments the cutaneous tissue site injury element is a chemical injury element. The kits for practicing the subject methods may further include a candidate agent; and an animal.

The kits of the subject methods may further include a cutaneous tissue site injury assessment element. Such an assessment element may include assaying the injured cutaneous tissue site for the release of at least one injury mediating chemicals, such as cytokines, neurotransmitters, growth factors, cytokines, or protaglandins. The assessment element may also include measuring vascular perfusion at the injured cutaneous tissue site, wherein such measurement may be performed by laser Doppler, for example, by scanning laser Doppler imaging. The assessment element may also include physical inspection of the injured cutaneous tissue site, wherein such inspection may include subjectively interrogating the animal regarding the injured cutaneous tissue site.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Systems

Also provided are systems that find use in practicing the subject methods, as described above. For example, in some embodiments, systems for practicing the subject methods may include a candidate analgesic agent, an element for locally administering said candidate agent to a tissue site, and an animal. In some embodiments the local administration element is a microdialysis device.

In other embodiments the system may further include a tissue site injury element. The tissue site injury element may be either a physical injury element, such an ultraviolet light source, or it may be a chemical injury element.

Systems of the subject methods may also include a tissue site injury assessment element. Such an assessment element may include assaying the injured tissue site for the release of at least one injury mediating chemicals, such as cytokines, neurotransmitters, growth factors, cytokines, or protaglandins. The assessment element may also include measuring vascular perfusion at the injured cutaneous tissue site, wherein such measurement may be performed by laser Doppler, for example, by scanning laser doppler imaging. The assessment element may also include physical inspection of the injured tissue site, wherein such inspection may include subjectively interrogating the animal regarding the injured tissue site.

The following examples are offered by way of illustration and not by way of limitation.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

I. Materials and Methods

A. Animals

Human

10 male and female adults, 18-70 yrs.

B. Injury of Cutaneous Tissue Site

Physical During the study a circular spot of 1.5 cm diameter will be irradiated on a subject's thigh. The spot will be irradiated with a UVB dose corresponding to 3 times the minimum effective dose to produce 1^{st} degree sunburn. Irradiation of a spot will take about 5 minutes. UVB burn will be provided by a calibrated UVB source previously used for inducing skin inflammation (Saalmann Multitester SBB LT 400, Saalmann GmbH, Herford, Germany). This device has been used in many human studies conducted by several laboratories and has an impecable safety record for doses up to eight time as high as suggested for this study. (1) The device is approved for clinical use, i.e. light therapy in dermatology, in Europe (http://www.saalmann.net/multi.html).

Chemical: Alternatively, a 1.5 cm diameter spot will be chemically treated on a subject's thigh. The vanilloid toxin, capsaicin, which provides the piquancy to peppers, will be topically applied to the skin spots (50 microliters, 1% solution in ethanol/water).

C. Delivery of Candidate Analgesic Agent

Microdialysis. A custom made microdialysis cathether will be threaded subcutaneously into the damaged skin area. This catheter can be perfused with Ringer's lactate at one of a range of speeds. In this case, the fluid will be perfused at a rate of 2-3 microliters/minute. Perfusate will be collected at the exit side of the catheter for analysis of biomolecules being released into the subcutaneous tissue. In addition, a known concentration of a candidate agent, for example, 1.0 micrograms/microliter of a COX-2 inhibitor is added to the perfusate input solution. In this way, the effects of the test agent on the release of biomarkers as well as effects on physical markers of inflammation (e.g., Doppler changes and pain sensitivity changes) can be determined.

D. Analysis

Subjective interrogation

Quantitative Sensory testing will be performed on subjects before and after application of injury and before and after application of test agent. This testing will include testing of force (mechanical) pain thresholds, touch thresholds, heat pain thresholds, and cold pain thresholds, as well as ratings of ongoing pain using standard methodologies.

Immuno-based assays (ELISA, Immunoprecip.)

Standard immuno-assays will be performed to determine the levels of various biomarkers in the perfusate exiting the catheter. These methods can include radioimmunoassay, ELISA, immunoprecipitation, or Bioplex methods. These assays will use widely available, commercial methods and assays. In this case, we will use Bioplex methods to simultaneously assay Interleukin 1-beta, Interleukin-6, tumor necrosis factor-alpha, nerve growth factor, substance P, calcitonin gene related peptide, and prostaglandin E2.

Biochemical assays

In some cases, biochemical assays, particularly high performance liquid chromatography (HPLC) can be used to assess certain biomarkers. In this case, we will use HPLC to measure perfusate levels of released amino acids, including aspartate and glutamate.

Laser Doppler

In some cases, we will assess the level of inflammation by measuring cutaneous blood flow by way of laser Doppler measurements. In this case, we will use scanning laser Doppler imaging to provide direct quantitation of the extent (area) of inflammamed tissue following injury, and following application of the test agent.

II. Results

Our results in this experiment will demonstrate a clear increase in cutaneous release of all of the above biomarkers, in pain sensitivity, and in area of inflammation following either UVB or capsaicin skin injury. Furthermore, application of the test COX-2 agent will substantially decrease the levels of the biomarkers, the area of inflammation, and of pain sensitivity of the injured areas.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method for evaluating the activity of a candidate anti-inflammatory, local anesthetic, or analgesic agent, said method comprising:
locally or systemically administering said candidate agent to a normal or injured cutaneous, joint, or muscular tissue site of an animal; and
assessing said injured tissue site to evaluate anti-inflammatory, local anesthetic, or analgesic activity of said candidate agents.

2. The method according to claim 1, wherein said candidate agent is locally administered by microdialysis.

3. The method according to claim 1, wherein said method further comprises injuring said tissue site prior to said locally administering step.

4. The method according to claim 3, wherein said injuring comprises physically injuring said tissue site.

5. The method according to claim 3, wherein said injuring comprises chemically injuring said tissue site.

6. The method according to claim 1, wherein said animal is a mammal.

7. The method according to claim 6, wherein said mammal is a human.

8. The method according to claim 1, wherein said candidate analgesic agent is a small molecule.

9. The method according to claim 1, wherein said assessing comprises assaying for the release of at least one injury mediating chemical from said site.

10. The method according to claim 9, wherein said at least one injury mediating chemical is a prostaglandin.

11. The method according to claim 1, wherein said assessing comprises measuring vascular perfusion at said site.

12. The method according to claim 11, wherein vascular perfusion at said site is measured by Scanning Laser Doppler Imaging.

13. The method according to claim 1, wherein said assessing comprises physical inspection of said injured tissue site.

14. The method according to claim 1, wherein said assessing comprises subjectively interrogating said animal.

15. A kit comprising:
a local administration element for locally administering a candidate agent to a tissue site; and
instructions for using said element in a method of evaluating the activity of a candidate agent.

16. The kit according to claim 15, wherein said local administration element is a microdialysis device.

17. The kit according to claim 15, wherein said kit further comprises a tissue site injury element.

18. The kit according to claim 17, wherein said injury element is a physical injury element.

19. The kit according to claim 17, wherein said injury element is a chemical injury element.

20. The kit according to claim 15, wherein said kit further comprises a cutaneous tissue site injury assessment element.

21. A system comprising:
(a) a candidate gent;
(b) an element for locally administering said candidate agent to a tissue site; and
(c) an animal.

22. The system according to claim 21, wherein said local administration element is a microdialysis device.

23. The system according to claim 22, wherein said system further comprises a tissue site injury element.

24. The system according to claim 23, wherein said injury element is a physical injury element.

25. The system according to claim 23, wherein said injury element is a chemical injury element.

26. The system according to claim 21, wherein said system further comprises a tissue site injury assessment element.
